(19) United States
(12) Patent Application Publication
(10) Pub. No.: US 2026/0114895 A1
(43) Pub. Date: Apr. 30, 2026

(54) PIPE SUPPORT DEVICE

(71) Applicant: INDUSTRIAL TECHNOLOGY RESEARCH INSTITUTE, Hsinchu (TW)

(72) Inventors: Hao-Yan WU, Taoyuan City (TW); Yan-Ting CHEN, Linluo Township (TW); An-Peng WANG, Kaohsiung City (TW); Yu-Hsien LIU, Tainan City (TW); Chien-Yu WU, Kouhu Township (TW)

(73) Assignee: INDUSTRIAL TECHNOLOGY RESEARCH INSTITUTE, Hsinchu (TW)

(21) Appl. No.: 19/001,876

(22) Filed: Dec. 26, 2024

(30) Foreign Application Priority Data
Oct. 28, 2024 (TW) 113141087

(51) Int. Cl. A61B 17/34; A61B 17/00; A61B 90/00; A61B 90/50

(52) U.S. Cl. CPC A61B 17/34; A61B 90/06; A61B 90/50; A61B 2017/00017; A61B 2017/00477; A61B 2017/00544; A61B 2017/00557; A61B 2090/064

(57) ABSTRACT

A pipe support device includes a pipe, an expandable component, a gas supply module and a gas pressure sensor. The expandable component is disposed outside the pipe. The gas supply module is connected to the expandable component and is configured to deliver a gas to the expandable component or to release the gas in the expandable component. The air pressure sensor is connected to the expandable component and is configured to sense a gas pressure of the expandable component.

This application claims the benefit of Taiwan application Serial No. 113141087, filed Oct. 28, 2024, the disclosure of which is incorporated by reference herein in its entirety.

### TECHNICAL FIELD

The disclosure relates in general to a pipe support device.

### BACKGROUND

A pipe support device may control a tool (for example, a needle or a cauterizer, etc.) to enter an interior of an object to detect a tissue inside the object. If abnormal tissue is found, the abnormal tissue may even be processed (for example, removed). However, depending on the location of the abnormal tissue, the needle may not be able to accurately target the abnormal tissue (for example, when the needle exerts force on the tissue, the position of the tool will be unstable). Therefore, proposing a technology that may improve the aforementioned problems is one of the goals of those in this technical field.

### SUMMARY

According to an embodiment, a pipe support device is provided. The pipe support device includes a pipe, an expandable component, a gas supply module and an expandable-component air pressure sensor. The expandable component is disposed outside the pipe. The gas supply module is connected to the expandable component and configured to deliver a gas to the expandable component or to pump the gas out of the expandable component. The expandable-component air pressure sensor is connected to the expandable component and configured to sense a gas pressure of the gas of the expandable element.

The above and other aspects of the disclosure will become better understood with regard to the following detailed description of the preferred but non-limiting embodiment(s). The following description is made with reference to the accompanying drawings.

### BRIEF DESCRIPTION OF THE DRAWINGS

FIG. 1A illustrates a schematic diagram of a pipe support device applied to a to-be-tested object according to an embodiment of the present invention;

FIG. 1B illustrates a schematic diagram of the pipe support device in FIG. 1A:

FIG. 1C illustrates a schematic diagram of a front view of a pipe support module of the pipe support device in FIG. 1A;

FIG. 2 illustrates a schematic diagram of a functional block of the pipe support device in FIG. 1A;

FIG. 3 illustrates a schematic diagram of the tool 105 in FIG. 1B puncturing the bronchus;

FIG. 4 illustrates a schematic diagram of a pipe support module according to another embodiment of the present disclosure;

FIG. 5 illustrates a schematic diagram of a pipe support module according to another embodiment of the present disclosure;

FIG. 6A illustrates a schematic diagram of the pipe and the end cap of the pipe support device in FIG. 1A (the tool and the airbag are not illustrated);

FIG. 6B illustrates a schematic diagram of perspective view of an end of the pipe support device in FIG. 1A;

FIGS. 6C and 6D illustrates a schematic diagram of the tool of the pipe support device in FIG. 6B being bent;

FIG. 7A illustrates a schematic diagram of the dimensions of the first airbag and the second airbag of the pipe support device in FIG. 6B;

FIG. 7B illustrates a schematic diagram of an embodiment when the volume of the first airbag in FIG. 7A increases;

FIG. 7C illustrates a schematic diagram of an embodiment when of the volume of the second airbag in FIG. 7A reduces;

FIG. 8A illustrates a schematic diagram of the pipe and the end cap of a pipe support device according to another embodiment of the present disclosure (the tool 105 and the airbag are not illustrated);

FIG. 8B illustrates a schematic diagram of a perspective view of the end of the pipe support device in FIG. 8A;

FIG. 8C illustrates a schematic diagram of a front view of the pipe support device in FIG. 8B;

FIG. 9A illustrates a schematic diagram of the pipe and the end cap of a pipe support device according to another embodiment of the present disclosure (the tool 105 and the airbag are not illustrated);

FIG. 9B illustrates a schematic diagram of a perspective view of the end of the pipe support device in FIG. 9A; and

FIG. 9C illustrates a schematic diagram of a front view of the pipe support device in FIG. 9B.

### DETAILED DESCRIPTION

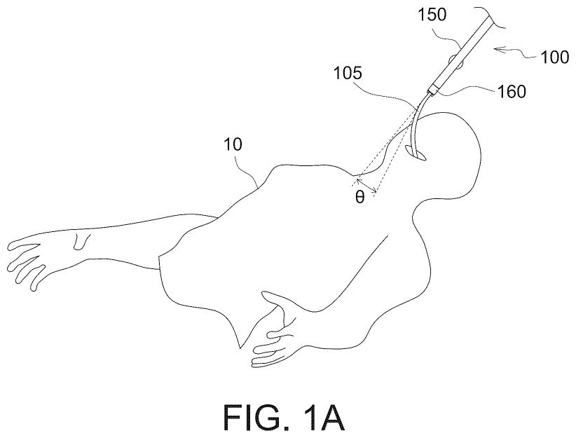

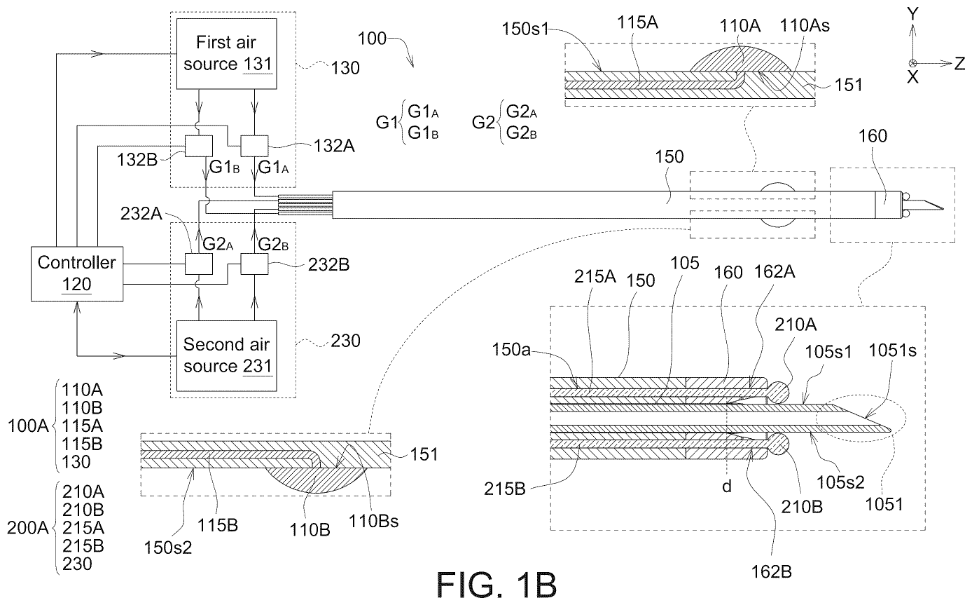

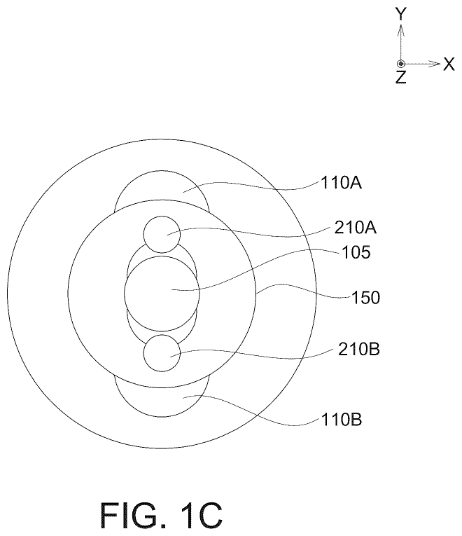

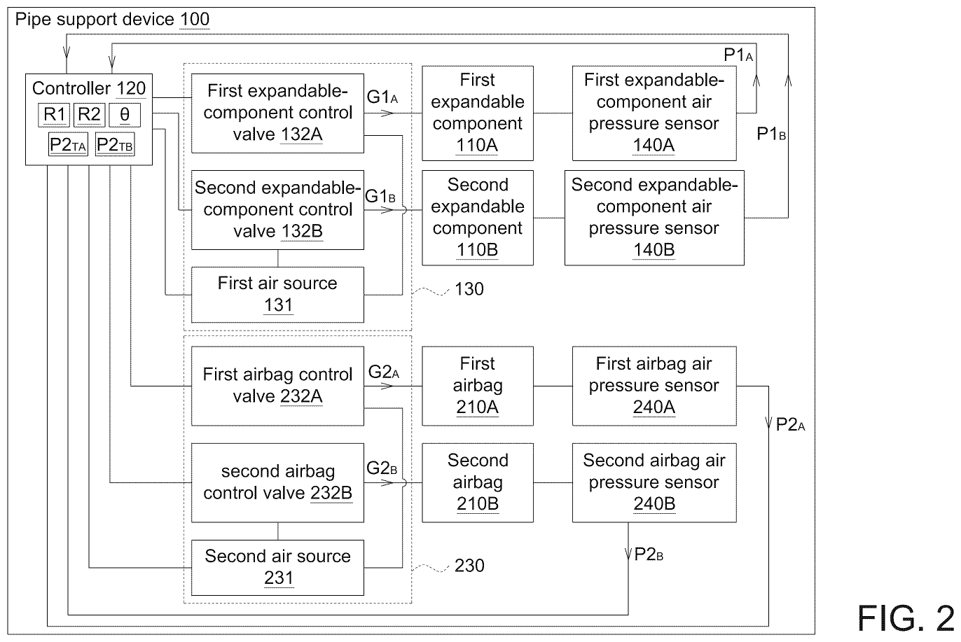

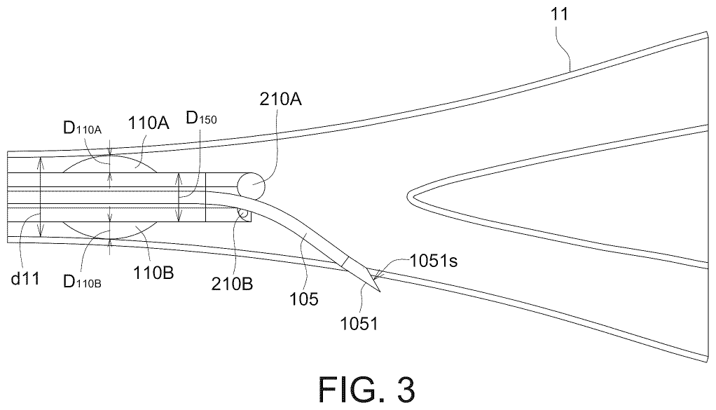

Referring to FIGS. 1A to 3, FIG. 1A illustrates a schematic diagram of a pipe support device 100 applied to a to-be-tested object 10 according to an embodiment of the present invention, FIG. 1B illustrates a schematic diagram of the pipe support device 100 in FIG. 1A, FIG. 1C illustrates a schematic diagram of a front view of a pipe support module 100A of the pipe support device 100 in FIG. 1A, FIG. 2 illustrates a schematic diagram of a functional block of the pipe support device 100 in FIG. 1A, and FIG. 3 illustrates a schematic diagram of the tool 105 in FIG. 1B puncturing the bronchus.

As illustrated in FIG. 1A, the pipe support device 100 may be applied in the medical field or industrial engineering field. In the medical field, the pipe support device 100 may be applied to a robot arm. The pipe support device 100 may control the movement of the tool 105, such as forward, backward, rotation and/or turning. The tool 105 is, for example, a needle, a cauterizer, a micro camera, a scraper (used to scrape off foreign matter, etc.) and other tools. The tool 105 may enter the interior of the to-be-tested object 10 to process the internal structure of the to-be-tested object 10. The to-be-tested object 10 may be a living body (for example, a human or an animal, or a natural hole or passage such as a bronchi or intestine in the living body) or a non-living body (for example, an object other than living things such as a building, a processing machine, a ground, an electronic device, a liquid channel, a gas channel, etc.). In the case of living body, internal structures include abnormal tissues such as tumors and polyps. Although not illustrated, the pipe support device 100 further includes a camera which may be disposed at a front end or an end cap 160 of the tool 105 to capture front images, where the images may be transmitted to the controller 120 (described later).

As illustrated in FIGS. 1B and 2, the pipe support device 100 includes the tool 105 (optionally), the controller 120, a pipe 150, an end cap 160, a pipe support module 100A and a tool attitude control module 200A. The pipe 150 has a hollow channel 150a, and the aforementioned tool 105 may pass through the hollow channel 150a of the pipe 150. The pipe support module 100A may support the pipe 150. As a result, when the tool 105 works in the to-be-tested object 10 (for example, punctures the bronchus), the pipe 150 of the pipe support module 100A may press against or support on the tissue of the to-be-tested object 10, and accordingly it may prevent the pipe 150 from retreating due to reaction force. The tool attitude control module 200A may control the steering of the tool 105.

As illustrated in FIGS. 1B and 2, the pipe support module 100A includes a plurality of expandable components (for example, a first expandable component 110A and a second expandable component 110B), a plurality of expandable-component gas delivery pipes (for example, a first expandable-component gas delivery pipe 115A and a second expandable-component gas delivery pipe 115B), a first gas supply module 130 and a plurality of expandable-component air pressure sensors (for example, a first expandable-component air pressure sensor 140A and the second expandable-component air pressure sensor 140B).

As illustrated in FIGS. 1B, 2 and 3, the first expandable component 110A and the second expandable component 110B are disposed outside the pipe 150. The first gas supply module 130 is connected to the expandable components (for example, the first expandable component 110A and the second expandable component 110B), and is configured to deliver a first gas G1 to the expandable components. The expandable-component air pressure sensor (for example, the first expandable-component air pressure sensor 140A and the second expandable-component air pressure sensor 140B) is connected to the expandable component and is configured to sense the pressure inside the gas pressure of the expandable component. In an embodiment, as illustrated in FIG. 3, when the tool 105 works (for example, punctures a cavity 11), the expandable component (for example, the first expandable component 110A and the second expandable component 110B) presses against an inner wall of the cavity 11 (e.g., bronchus), and thus it may prevent the pipe 150 from retreating due to reaction force. In addition, the expandable component properly presses against the inner wall of the bronchus and does not cause the bronchial dilation of the cavity 11. Furthermore, the expandable component in an embodiment of the present disclosure is not used to expand the cavity 11, but simply abuts against the inner wall of the cavity 11.

As illustrated in FIGS. 1B and 3, the tool 105 has a puncturing 1051, and the puncturing tip 1051 has an inclined surface 1051s. The inclined surface 1051s and the expandable component are disposed on the same side. For example, the inclined surface 1051s and the first expandable component 110A are disposed on the same side. For example, the tool 105 has a first lateral surface 105s1 and a second lateral surface 105s2 opposite to the first side 105s1, wherein the inclined surface 1051s extends obliquely from the second lateral surface 105s2 to the first lateral surface 105s1. There is an acute angle between the inclined surface 1051s and the second lateral surface 105s2, so that a facing direction of the inclined surface 1051s is substantially the same as a facing direction of the first lateral surface 105s1. As a result, the inclined surface 1051s and the first expandable component 110A are disposed substantially on the same side. Since the inclined surface 1051s and the first expandable component 110A are disposed on the same side, when the tool 105 punctures the inner wall of the cavity 11 toward the second lateral surface 105s2 (downward), a reaction force will be generated toward the first lateral surface 105s1 (upward), and the expanded first expandable component 110A may resist such reaction force to prevent the pipe support device 100 from retreating or slipping, thereby increasing the puncture stability and/or accuracy of the tool 105 (if the pipe support device is deviated, it may cause the tool to puncture inaccurately).

As illustrated in FIGS. 1B and 1C, the number of expandable components of the pipe support module 100A is, for example, two, but the embodiment of the present disclosure is not limited to this. The first expandable component 110A and the second expandable component 110B are, for example, disposed oppositely, that is, an included between the first expandable component 110A and the second expandable component 110B is, for example, 180 degrees. In another embodiment, an included between the first expandable component 110A and the second expandable component 110B may be different from 180 degrees (that is, the included angle is not equal to 180 degrees). In addition, the first expandable component 110A has a first connection surface 110As, wherein the first connection surface 110As may be connected to a first outer wall surface 150s1 of the pipe 150. The first connection surface 110As is fixed to the first outer wall surface 150s1 of the pipe 150 by, for example, adhesive means. Similarly, the second expandable component 110B has a second connection surface 110Bs, wherein the first connection surface 110As may be connected to a second outer wall surface 150s2 of the pipe 150. The second connection surface 110Bs is fixed to the second outer wall surface 150s2 of the pipe 150 by, for example, adhesive means. Since the connection surface of the expandable component is fixed to the outer wall surface of the pipe 150, the stability of the expandable component against the inner wall of the cavity 11 may be increased.

As illustrated in FIG. 1B, the first expandable-component gas delivery pipe 115A may be embedded in a pipe body 151 of the pipe 150, wherein the pipe body 151 is the solid (or physical) material of the pipe 150. Similarly, the second expandable-component gas delivery pipe 115B may be embedded in the pipe body 151 of the pipe 150. In another embodiment, the first expandable-component gas delivery pipe 115A may be disposed in the hollow channel 150a of the pipe 150 and/or the second expandable-component gas delivery pipe 115B may be disposed in the hollow channel 150a of the pipe 150.

As illustrated in FIGS. 1B and 2, the first gas supply module 130 includes a first air source 131 and a plurality of control valves (for example, a first expandable-component control valve 132A and a second expandable-component control valve 132B). The first air source 131 is connected to the first expandable-component control valve 132A and the second expandable-component control valve 132B. The first gas G1 supplied by the first air source 131 may be delivered to the expandable component through the control valve. The controller 120 is electrically connected to the first air source 131 to control the air pressure of the first gas G1 delivered to the expandable component. The aforementioned first expandable-component gas delivery pipe 115A connects the first expandable component 110A with the first expandable-component control valve 132A. The first gas G1a may be delivered to the first expandable component 110A thorough the first expandable-component control valve 132A and the first expandable-component gas delivery pipe 115A. The aforementioned second expandable-component gas delivery pipe 115B connects the second expandable component 110B with the second expandable-component control valve 132B. The second gas GIB may be delivered to the second expandable component 110B through the second expandable-component control valve 132B and the second expandable-component gas delivery pipe 115B.

As illustrated in FIGS. 1B and 2, the first expandable-component control valve 132A is connected to the first expandable component 110A to close or open a channel of the first gas GIA delivered to the first expandable component 110A. The second expandable-component control valve 132B is connected to the second expandable component 110B to close or open a channel of the second gas G2_{B} delivered to the second expandable component 110B. The controller 120 is electrically connected to the first expandable-component control valve 132A and the second expandable-component control valve 132B to control these control valves to close or open. In addition, the controller 120 may control the opening of the control valve to control the amount of the delivered gas, thereby controlling the air pressure of the expandable component (controlling the volume (or outer diameter) of the expandable component). In addition, the controller 120 may control the first air source 131 to pump the gas out of the individual expandable component to reduce the volume (or outer diameter) of the expandable components.

As illustrated in FIGS. 1B and 3, the controller 120 is, for example, a physical circuit formed using at least one semiconductor process, such as a semiconductor chip or a semiconductor package. The controller 120 is electrically connected to the first gas supply module 130 and is configured to control the first gas supply module 130 to supply the first gas G1 to the expandable component according to an inner diameter d11 of the cavity 11 of the to-be-tested object 10. For example, an expansion size of the expandable component may be determined by the amount of gas in the expandable component. For example, an outer diameter of the expandable component is proportional to the amount of gas in the expandable component. The controller 120 may control the first gas supply module 130 to supply an appropriate amount of the first gas G1 to the expandable component according to the inner diameter d11 of the cavity 11, so that the expandable component expands to an appropriate outer diameter for proper abuts against the inner wall of the cavity 11 of the to-be-tested object 10. Taking the first expandable component 110A and the second expandable component 110B as an example, the expanded first expandable component 110A has a first outer diameter D_{110A}, and the expanded second expandable component 110B has a third outer diameter D_{110A}, the sum of the two outer diameters D_{110B}, the first outer diameter D_{110A}, the second outer diameter D_{110B} and the outer diameter D₁₅₀ of the pipe 150 is substantially equal to or greater than the inner diameter d11 of the cavity 11. Although the sum of the first outer diameter D_{110A}, the second outer diameter D_{110B} and the outer diameter D₁₅₀ of the pipe 150 may be greater than the inner diameter d11 of the cavity 11, it is not enough to expand the cavity 11.

In an embodiment, the controller 120 may provide the first gas G1 corresponding to the inner diameter d11 of the cavity 11 to the expandable component according to a corresponding relationship R1 between an expandable-component gas pressure and an inner diameter of the cavity. The corresponding relationship between the expandable-component gas pressure and the inner diameter of the cavity may be obtained in advance by simulation or experiment, and stored in the controller 120 or in a memory (not illustrated) that the controller 120 may access. For further example, the controller 120 is further configured to: according to the corresponding relationship R1, determine whether the air pressure in the expandable component has reached a corresponding air pressure, wherein the corresponding air pressure is the air pressure that just abuts against the inner wall of the cavity 11, or the expandable component may abut against the inner wall of the cavity 11 and resist the air pressure of the puncture force. When the air pressure in the expandable component does not reach the corresponding air pressure, the first gas supply module 130 is controlled to continue to supply the first gas G1 to the expandable component; and when the air pressure in the expandable component reaches the corresponding air pressure, the first gas supply module 130 is controlled to stop supplying the first gas G1 to the expandable component. The aforementioned corresponding relationship R1 is, for example, a table, a mathematical equation, etc. Such correspondence relationship R1 may be obtained in advance by experiments or software simulation, and then stored in the controller 120, or in a memory or storage electrically connected to the controller 120.

As illustrated in FIGS. 1B and 2, the expandable-component air pressure sensors are connected to these expandable components. For example, the first expandable-component air pressure sensor 140A is connected to the first expandable component 110A, and the second expandable-component air pressure sensor 140B is connected to the second expandable component 110B. Each expandable-component air pressure sensor is configured to sense a measured air pressure value of the corresponding expandable component and transmit the measured air pressure value to the controller 120. For example, the first expandable-component air pressure sensor 140A may sense a first measured air pressure value PIA of the first expandable component 110A and transmit the first measured air pressure value PIA to the controller 120, while the second expandable-component air pressure sensor 140B may sense a second measured air pressure value PIB of the second expandable component 110B and transmit the first measured air pressure value PIA to the controller 120.

In the present embodiment, the first expandable-component air pressure sensor 140A may be disposed within the first expandable component 110A for sensing the air pressure value of the first expandable component 110A, and the second expandable-component air pressure sensor 140B may be disposed within the second expandable component 110B to sense the air pressure value of the second expandable component 110B. In another embodiment, the first expandable-component air pressure sensor 140A may be disposed in the first expandable-component gas delivery pipe 115A for sensing the air pressure value of the pipeline connected to the first expandable component 110A. The second expandable-component air pressure sensor 140B may be disposed within the second expandable-component gas delivery pipe 115B for sensing the air pressure value of the pipeline connected to the second expandable component 110B. In other embodiment, the first expandable-component air pressure sensor 140A may be disposed within the first expandable-component control valve 132A for sensing the air pressure value delivered to the first expandable component 110A, and the second expandable-component air pressure sensor 140B may be disposed within the second expandable-component control valve 132B for sensing the air pressure value delivered to the second expandable component 110B. However, the arrangement position of the expandable-component air pressure sensor is not limited by the embodiments herein, as long as the measured air pressure value may reflect the actual air pressure of the airbag.

In the present embodiment, the expandable component is, for example, an airbag. The end cap 160 is connected to the pipe 150, and the expandable component is spaced apart from the end cap 150 by a distance. Furthermore, the first expandable-component air pressure sensor 140A and the second expandable-component air pressure sensor 140B do not contact the end cap 160 but are spaced apart from the end cap 160.

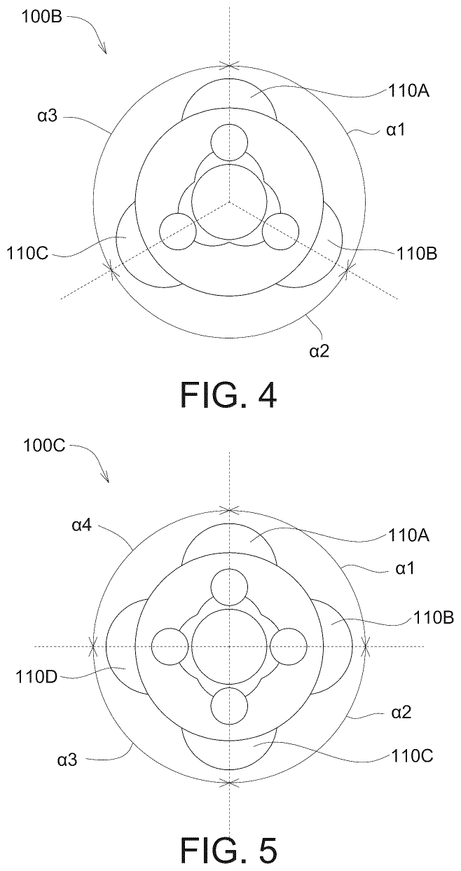

Referring to FIGS. 4 and 5, FIG. 4 illustrates a schematic diagram of a pipe support module 100B according to another embodiment of the present disclosure, and FIG. 5 illustrates a schematic diagram of a pipe support module 100C according to another embodiment of the present disclosure.

As illustrated in FIG. 4, the number of the expandable components of the pipe support module 100B is three, for example, the first expandable component 110A, the second expandable component 110B and the third expandable component 110C, wherein the first expandable component 110A, the second expandable component 110B and the third expandable component 110C are disposed by way of, for example, equal central angle. For example, an included angle α1 between the first expandable component 110A and the second expandable component 110B is 120 degrees, an included angle α2 between the second expandable component 110B and the third expandable component 110C is 120 degrees, and an included angle α3 between the third expandable component 110C and the first expandable component 110A is 120 degrees. However, in another embodiment, two of the included angle α1, the included angle α2 and the included angle α3 may be different.

As illustrated in FIG. 5, the number of expandable components of the pipe support module 100C is 4, for example, the first expandable component 110A, the second expandable component 110B, the third expandable component 110C and the fourth expandable component 110D, wherein the first expandable component 110A, the second expandable component 110B, the third expandable component 110C and the fourth expandable component 110D are disposed by way of, for example, equal central angle. For example, an included angle α1 between the first expandable component 110A and the second expandable component 110B is 90 degrees, an included angle α2 between the second expandable component 110B and the third expandable component 110C is 90 degrees, an included angle α3 between the third expandable component 110C and the fourth expandable component 110D is 90 degrees, and an included angle α4 between the fourth expandable component 110D and the first expandable component 110A is 90 degrees. However, in another embodiment, two of the included angle α1, the included angle α2, the included angle α3 and the included angle α4 may be different.

As illustrated in FIGS. 1B and 2, the tool attitude control module 200A includes a plurality of airbags (for example, a first airbag 210A and a second airbag 210B), and a plurality of airbag gas delivery pipes (for example, a first airbag gas delivery pipe 215A and a second airbag gas delivery pipe 215B), a second gas supply module 230 and a plurality of airbag gas pressure sensors (for example, as illustrated in FIG. 2, a first airbag gas pressure sensor 240A and a second airbag gas pressure sensor 240B). The aforementioned expandable components and the airbags of the tool posture control module 200A may be made of the same material.

As illustrated in FIGS. 1B and 2, the second gas supply module 230 is connected to the first airbag 210A and the second airbag 210B. The controller 120 is electrically connected to the second gas supply module 230. The tool 105 is disposed between the first airbag 210A and the second airbag 210B. The controller 120 is electrically connected to the second gas supply module 230 and is configured to control the second gas supply module 230 to supply a first gas G2_{A} and a second gas G2_{B} to the first airbag 210A and the second airbag 210B respectively according to a target bending angle value θ of the tool 105 (the target bending angle value θ is illustrated in FIG. 1A). As a result, the size (e.g., volume) of the airbag may be individually controlled through the gas for controlling (or adjusting) the bending angle (e.g., a steering and/or a turning angle) of the tool 105. In other words, the posture of the tool 105 in the embodiment of the present disclosure is controllable. Therefore, regardless of the position of the abnormal tissue relative to the tool 105, the abnormal tissue may be treated by the bending of the tool 105.

As illustrated in FIG. 1B, the first airbag 210A and the second airbag 210B are located outside the pipe 150 and the end cap 160, and leans on the end cap 160.

As illustrated in FIGS. 1B and 2, the controller 120 is configured to control the second gas supply module 230 to supply the first gas G2_{A} with a first output air pressure value to the first airbag 210A according to the target bending angle value θ, and supply the second gas G2_{B} with a second output air pressure value to the second airbag 210B, wherein the first output air pressure value and the second output air pressure value may be equal or different. By controlling the output pressure value of the gas, the volume of the airbag may be controlled, thereby controlling the bending mode of the tool 105.

As illustrated in FIGS. 1B and 2, the second gas supply module 230 includes a second air source 231 and a plurality of control valves (for example, a first control valve 232A and a second control valve 232B). The second air source 231 is connected to the first control valve 232A with the second control valve 232B, and the second gas G2 supplied by the second gas source 231 may be delivered to the airbag through the control valve. The controller 120 is electrically connected to the second air source 231 to control the gas pressure delivered to the control valve.

As illustrated in FIGS. 1B and 2, the first control valve 232A is connected to the first airbag 210A to be turned off or turned on for connection or disconnection of the first gas G2_{A} delivered to the first airbag 210A. The second control valve 232B is connected to the second airbag 210B to be turned off or turned on for connection or disconnection of the second gas G2_{B} delivered to the second airbag 210B. The controller 120 is electrically connected to the first control valve 232A and the second control valve 232B to control the control valves to be turned on or turned off. In addition, the controller 120 may control an opening level of the control valve for controlling the output gas pressure value delivered to the airbag. In addition, the controller 120 may control the second air source 231 to pump the gas out of the individual airbag to reduce the volume (or radius) of the airbag.

As illustrated in FIGS. 1A, 1B and 2, the controller 120 is further configured to obtain a target gas pressure value corresponding to the target bending angle value θ according to a corresponding relationship R2 between a plurality of bending angle values and a plurality of target gas pressure values. For example, in order to realize that the tool 105 presenting the target bending angle value θ, the controller 120 may obtain the gas pressure value of the first airbag 210A corresponding to the target bending angle value θ as a first target gas pressure value P2_{TA} and obtain the gas pressure value of the second airbag 210B corresponding to the target bending angle value θ as a second target gas pressure value P2_{TB} according to the aforementioned corresponding relationship. Therefore, the controller 120 may control the second air source 231, the first control valve 232A and/or the second control valve 232B, so that the gas pressure value of the first airbag 210A is the first target gas pressure value P2_{TA} and the gas pressure value of the second airbag 210B is the second target gas pressure value P2_{TB}. The aforementioned corresponding relationship R2 is, for example, a table, a mathematical equation, etc. This corresponding relationship R2 may be obtained in advance through experiments or software simulation, and then stored in the controller 120, or in a memory or storage electrically connected to the controller 12.

As illustrated in FIGS. 1B and 2, the airbag gas pressure sensors are connected to the airbags. For example, the first airbag gas pressure sensor 240A is connected to the first airbag 210A, and the second airbag gas pressure sensor 240B is connected to the second airbag 210B. Each airbag gas pressure sensor is configured to sense a measured gas pressure value of the corresponding airbag, and transmit the measured gas pressure value to the controller 120. For example, the first airbag gas pressure sensor 240A may sense a first measured gas pressure value P2_{A} of the first airbag 210A and transmit the first measured gas pressure value P2_{A} to the controller 120, while the second airbag gas pressure sensor 240B may sense a second measured gas pressure value P2_{B} of the second airbag 210B and transmit the second measured gas pressure value P2_{B} to the controller 120.

In the present embodiment, the first airbag gas pressure sensor 240A may be disposed within the first airbag 210A to sense the gas pressure value of the first airbag 210A, and the second airbag gas pressure sensor 240B may be disposed within the second airbag 210B to sense the gas pressure value of the second airbag 210B. In another embodiment, the first airbag gas pressure sensor 240A may be disposed within the first airbag gas delivery pipe 215A to sense the gas pressure value of the pipe connected to the first airbag 210A, and the second airbag gas pressure sensor 240B may be disposed within the second airbag gas delivery pipe 215B to sense the gas pressure value of the pipe connected to the second airbag 210B. In other embodiments, the first airbag gas pressure sensor 240A may be disposed on the first airbag control valve 232A to sense the gas pressure value delivered to the first airbag 210A, and the second airbag gas pressure sensor 240B may be disposed on the second airbag control valve 232B to sense the gas pressure value delivered to the second airbag 210B. However, the arrangement position of the airbag gas pressure sensor is not limited by the embodiments herein, as long as the gas pressure value measured by the airbag gas pressure sensor may reflect the actual gas pressure of the airbag.

The controller 120 is further configured to control the second gas supply module 230 to adjust the output value of the gas according to measured gas pressure values. Furthermore, the controller 120 is further configured to: determine whether a difference between each measured gas pressure value and the corresponding target gas pressure value is outside an error range; and control the second gas supply module to adjust the output value of the gas according to the measured gas pressure value whose difference is outside the error range, so that the difference falls within the error range.

For example, the controller 120 is further configured to: determine whether a first difference between the first measured gas pressure value P2_{A} of the first airbag 210A and the first target gas pressure value P2_{TA} is outside the error range; and control the second gas supply module 230 to adjust a first output value of the first gas G2_{A} according to the first measured gas pressure value P2_{A} whose first difference is outside the error range, so that the first difference falls within the error range. For example, when the first measured gas pressure value P2_{A} is greater than the first target gas pressure value P2_{TA}, the first output value of the first gas G2_{A} is reduced until the first difference falls within the error range. When the first measured gas pressure value P2_{A} is less than the first target gas pressure value P2_{TA}, the first output value of the first gas G2_{A} is increased until the first difference falls within the error range. Similarly, the controller 120 is further configured to: determine whether a second difference between the second measured gas pressure value P2_{B} of the second airbag 210B and the second target gas pressure value P2_{TB} is outside the error range; and control the second gas supply module 230 to adjust a second output value of the second gas G2_{B} according to the second measured gas pressure value P2_{B} whose second difference is outside the error range, so that the second difference falls within the error range. For example, when the second measured gas pressure value P2_{B} is greater than the second target gas pressure value P2_{TB}, the second output value of the second gas G2_{B} is reduced until the second difference falls within the error range. When the second measured gas pressure value P2_{B} is less than the second target gas pressure value P2_{TB}, the second output value of the second gas G2_{B} is increased until the second difference falls within the error range.

In addition, the controller 120 is further configured to determine a force-applied direction of the tool 105 according to the measured gas pressure values. For example, when the tool 105 is subjected to an external force, the corresponding airbag is squeezed. Therefore, through changes in the gas pressure value of the airbag, the force-applied direction of the tool 105 may be known.

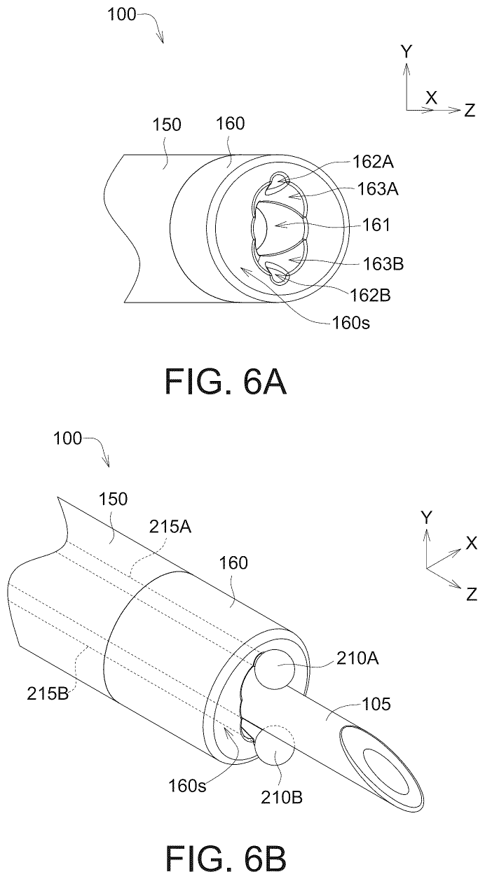

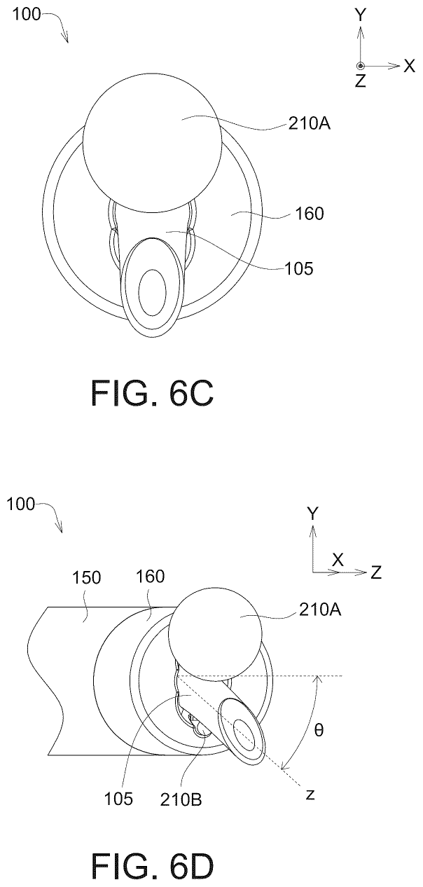

Referring to FIGS. 6A to 6D, FIG. 6A illustrates a schematic diagram of the pipe 150 and the end cap 160 of the pipe support device 100 in FIG. 1A (the tool 105 and the airbag are not illustrated), FIG. 6B illustrates a schematic diagram of perspective view of an end of the pipe support device 100 in FIG. 1A, and FIGS. 6C and 6D illustrates a schematic diagram of the tool 105 of the pipe support device 100 in FIG. 6B being bent.

As illustrated in FIG. 6A, the first airbag gas delivery pipe 215A and the second airbag gas delivery pipe 215B may be embedded in a pipe body 151 of the pipe 150. The end cap 160 is disposed at the end of the pipe 150 and has an end surface 160s, a tool channel 161 and a plurality of airbag gas delivery pipe channels (for example, a first airbag gas delivery pipe channel 162A and a second airbag gas delivery pipe channel 162B), wherein the tool channel 161 and the airbag gas delivery pipe channels extend inwardly from the end face 160s.

As illustrated in FIGS. 1B and 6A to 6B, the tool 105 enters and exits the end cap 160 through the tool channel 161. Each airbag gas delivery pipe is connected to the corresponding airbag through the corresponding airbag gas delivery pipe channel. For example, the first airbag gas delivery pipe 215A is connected to the first airbag 210A through the first airbag gas delivery pipe channel 162A, and the second airbag gas delivery pipe 215B is connected to the second airbag 210B through the second airbag gas delivery pipe channel 162B.

As illustrated in FIG. 6A, the end cap 160 further has a plurality of position-limiting grooves (for example, a first position-limiting groove 163A and a second position-limiting groove 163B). Each position-limiting groove communicates with the tool channel 161 and extends along a movement direction to provide a receiving space for the tool 105 along a movement direction, thereby providing a degree of freedom (DoF) of movement for the tool 105 along the movement direction. For example, the first position-limiting groove 163A extends from the tool channel 161 toward +Y-axis to allow the tool 105 to bend along+Y-axis (that is, the tool 105 bends around −X-axis), while the second position-limiting groove 163B extends from the tool channel 161 toward −Y-axis to allow the tool 105 to bend along the −Y-axis (that is, the tool 105 bending around +X-axis), as illustrated in FIGS. 6C and 6D. The z-axis in FIG. 6D is, for example, a central axis of the tool 105, which may represent a bending direction of the tool 105. The aforementioned target bending angle value θ is, for example, the angle between Z-axis and z-axis.

As illustrated in FIGS. 6C and 6D, when the volume of the first airbag 210A is greater than the volume of the second airbag 210B, the tool 105 is squeezed to bend in −Y-axis. In another embodiment, although not illustrated, when the volume of the second airbag 210B is greater than the volume of the first airbag 210A, the tool 105 is squeezed to bend in +Y-axis. In summary, the pipe support device 100 of the embodiment of the present disclosure may provide the tool 105 with the degree of freedom to bend in +/−Y-axis. In addition, although not illustrated, the tool 105 may rotate around Z-axis, so that the z-axis of the tool 105 may rotate around Z-axis.

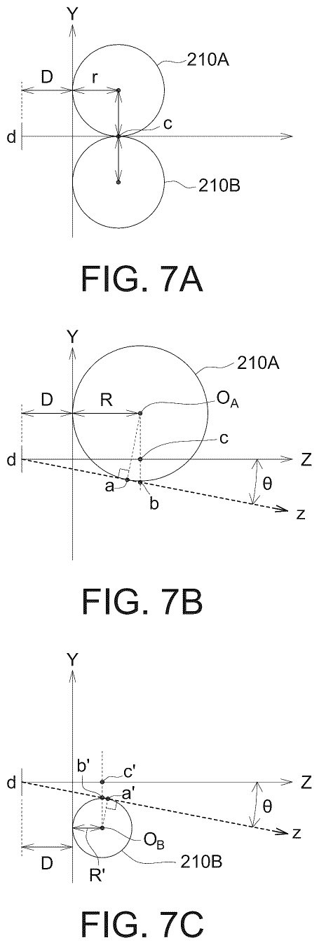

Referring to FIGS. 7A to 7C, FIG. 7A illustrates a schematic diagram of the dimensions of the first airbag 210A and the second airbag 210B of the pipe support device 100 in FIG. 6B, FIG. 7B illustrates a schematic diagram of an embodiment when the volume of the first airbag 210A in FIG. 7A increases, and FIG. 7C illustrates a schematic diagram of an embodiment when of the volume of the second airbag 210B in FIG. 7A reduces.

As illustrated in FIG. 7A, a position d is also illustrated in FIG. 1B, which is substantially a connection between the position-limiting grooves (the first position-limiting groove 163A and the second position-limiting groove 163B) and the tool channel 161. The tool 105 has bending freedom only in the position d. The Y-axis in FIG. 7A is the end surface 160s (the end surface 160s is illustrated in FIG. 6A) of the end cap 160. A dimension D is a distance along Z-axis between the position d and the end face 160s, a radius r is a distance along Z-axis between the end face 160s and a center point of the airbag, and a point c is a contact point between the two airbags.

As illustrated in FIG. 7B, when the volume of the first airbag 210A becomes greater, the radius increases to R from r. Based on the premise that Δdcb˜ΔO_{A}ab, ∠bdc=∠bO_{A}a=θ and b̅c̅=b̅O̅_̅{̅A̅}̅−O̅_̅{̅A̅}̅C̅, the following formula (1) may be derived. The symbol “˜” illustrated above is an “approximate symbol” in mathematics. The controller 120 may control the second gas supply module 230 to supply the first gas G2_{A} to the first airbag 210A according to formula (1), so that the first airbag 210A has the radius R to achieve the bending angle value of the tool 105 being the target bending angle value θ.

$$R = ( D sin θ + r cos θ ) / ( 1 - sin θ ) \qquad ( 1 )$$

As illustrated in FIG. 7C, when the volume of the second airbag 110B becomes smaller, the radius becomes smaller to R′ from r. Based on Δdc′b′˜ΔO_{B}a′b′, ∠b′dc′=∠b′O_{B}a′=θ and

$$\overline{{b}^{′} {c}^{′}} = \overline{{O}_{B} {c}^{′}} - \overline{{b}^{′} {O}_{B}} ,$$

the following formula (2) may be derived. The controller 120 may control the second gas supply module 230 to supply the second gas G2_{B} to the second airbag 210B according to formula (2), so that the second airbag 210B has the radius R′ to achieve the bending angle value of the tool 105 being the target bending angle value θ.

$${R}^{′} = ( r cos θ + D sin θ ) / ( 1 - sin θ ) \qquad ( 2 )$$

The formulas (1) and (2) are applicable formulas for the two airbags. In another embodiment, an applicable formula for N airbags may be derived by using similar principles.

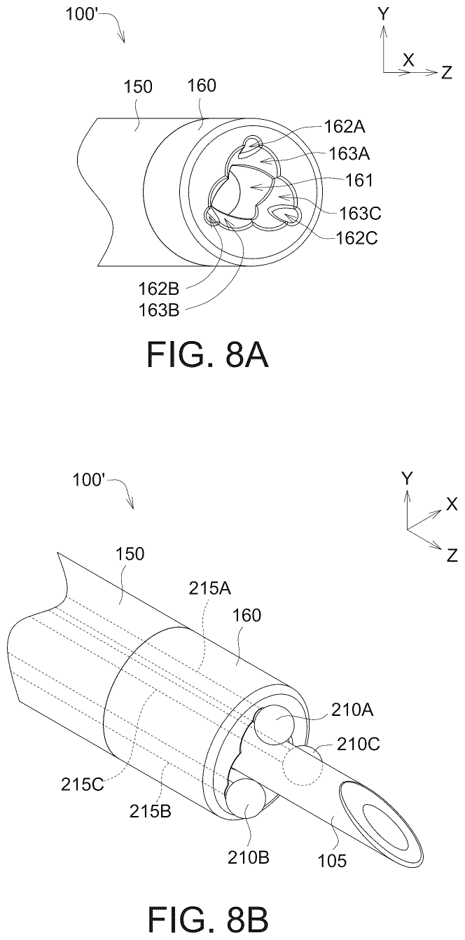

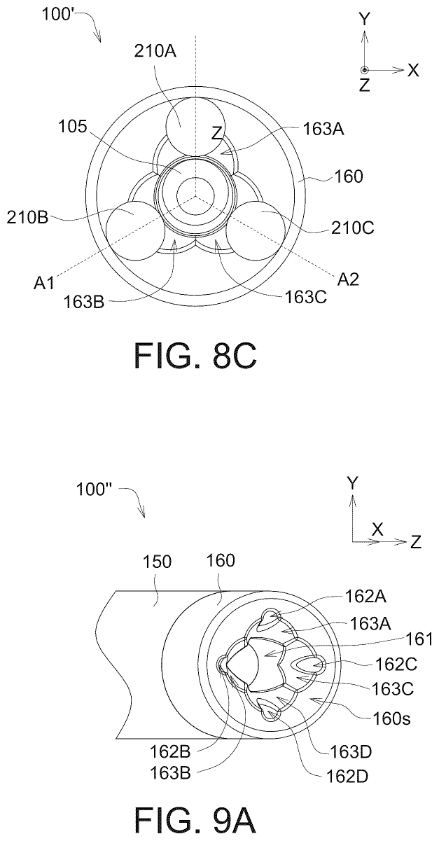

Referring to FIGS. 8A to 8C, FIG. 8A illustrates a schematic diagram of the pipe 150 and the end cap 160 of a pipe support device 100′ according to another embodiment of the present disclosure (the tool 105 and the airbag are not illustrated), FIG. 8B illustrates a schematic diagram of a perspective view of the end of the pipe support device 100′ in FIG. 8A, and FIG. 8C illustrates a schematic diagram of a front view of the pipe support device 100′ in FIG. 8B.

As illustrated in FIGS. 8A to 8B, the pipe support device 100′ includes the tool 105, the controller 120, the pipe 150, the end cap 160, the pipe support module 100A and the tool attitude control module 200B. The tool attitude control module 200B includes a plurality of airbags (for example, the first airbag 210A, the second airbag 210B and a third airbag 210C), a plurality of airbag gas delivery pipes (for example, the first airbag gas delivery pipe 215A, the second airbag gas delivery pipe 215B and a third airbag gas delivery pipe 215C), the second gas supply module (not illustrated) and a plurality of airbag air pressure sensors (not illustrated).

The tool attitude control module 200B includes the features (for example, structure, connection relationship, etc.) the same as or similar to that of the aforementioned tool attitude control module 200A, and one of the differences is that the number of the airbag gas delivery pipes of the tool attitude control module 200B is different.

As shown in FIGS. 8A and 8B, the first airbag gas delivery pipe 215A, the second airbag gas delivery pipe 215B and the third airbag gas delivery pipe 215C may be embedded in the pipe body 151 of the pipe 150. The end cap 160 is disposed at the end of the pipe 150 and has an end surface 160s, the tool channel 161 and a plurality of the airbag gas delivery pipe channels (for example, the first airbag gas delivery pipe channel 162A, the second airbag gas delivery pipe channel 162B and a third airbag gas delivery pipe channel 162C), wherein the tool channel 161 and the airbag gas delivery pipe channels extend inwardly from the end surface 160s.

As illustrated in FIGS. 8A and 8B, the tool 105 enters and exits the end cap 160 through the tool channel 161. Each airbag gas delivery pipe is connected to the corresponding airbag through the corresponding airbag gas delivery pipe channel. For example, the first airbag gas delivery pipe 215A is connected to the first airbag 210A through the first airbag gas delivery pipe channel 162A, the second airbag gas delivery pipe 215B is connected to the second airbag 210B through the second airbag gas delivery pipe channel 162B, and the third airbag gas delivery pipe 215C is connected to the third airbag 210C through the third airbag gas delivery pipe channel 162C.

In the present embodiment, the second gas supply module includes the second air source 231 and a plurality of control valves. The control valves of the second gas supply module in the present embodiment have the features same as or similar to that of the control valves of the second gas supply module 230 as described above, and it will not be repeated again here. The number of the control valves of the second gas supply module is equal to the number of the airbags in the present embodiment.

As illustrated in FIGS. 8A and 8C, the end cap 160 further has a plurality of the position-limiting grooves (for example, the first position-limiting groove 163A, the second position-limiting groove 163B and a third position-limiting groove 163C). Each position-limiting groove communicates with the tool channel 161 and extends along the movement direction to provide the tool 105 with a degree of freedom of movement along the movement direction. For example, the first position-limiting groove 163A extends from the tool channel 161 in +Y-axis to allow the tool 105 to bend in +Y-axis. The second position-limiting groove 163B extends from the tool channel 161 in a first axial direction A1 to allow the tool 105 to bend in the first axial direction A1. The third position-limiting groove 163C extends from the tool channel 161 in a second axial direction A2 to allow the tool 105 to move in the second axial direction A2. In an embodiment, a central angle between the first axial direction A1 and Y-axis is, for example, 120 degrees, and a central angle between the first axial direction A1 and the second axis A2 is, for example, 120 degrees. However, such embodiment in the present disclosure is not limited to this.

In an embodiment, the radius (or volume) of the first airbag 210A, the radius (or volume) of the second airbag 210B, and/or the radius (or volume) of the third airbag 210C may be controlled to control the bending direction and/or the bending angle of the tool 105.

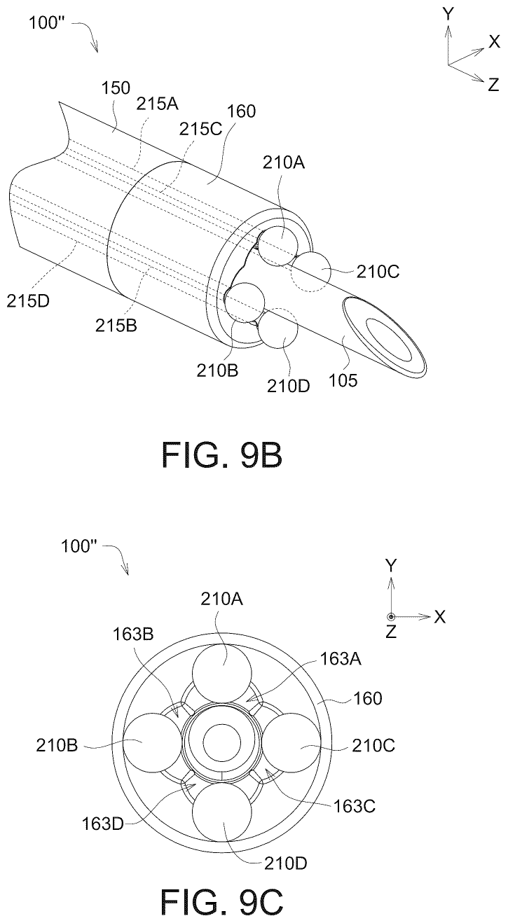

Referring to FIGS. 9A to 9C, FIG. 9A illustrates a schematic diagram of the pipe 150 and the end cap 160 of a pipe support device 100″ according to another embodiment of the present disclosure (the tool 105 and the airbag are not illustrated), FIG. 9B illustrates a schematic diagram of a perspective view of the end of the pipe support device 100″ in FIG. 9A, and FIG. 9C illustrates a schematic diagram of a front view of the pipe support device 100″ in FIG. 9B.

As illustrated in FIGS. 9A to 9B, the pipe support device 100″ includes the tool 105 (optional), the controller 120, the pipe 150, the end cap 160, the pipe support module 100A and a tool attitude control module 200C. The tool attitude control module 200C includes a plurality of airbags (for example, the first airbag 210A, the second airbag 210B, the third airbag 210C and a fourth airbag 210D), and a plurality of airbag gas delivery pipes (for example, the first airbag gas delivery pipe 215A, the second airbag gas delivery pipe 215B, the third airbag gas delivery pipe 215C and a fourth airbag gas delivery pipe 215D), the second gas supply module (not illustrated) and a plurality of airbag air pressure sensors (not illustrated).

The tool attitude control module 200C includes the technical features (for example, structure, connection relationship, etc.) the same as or similar to that of the aforementioned tool attitude control module 200B. One of the differences is that the number of the airbag gas delivery pipes of the tool attitude control module 200C is different.

As illustrated in FIGS. 9A and 9B, the first airbag gas delivery pipe 215A, the second airbag gas delivery pipe 215B, the third airbag gas delivery pipe 215C and the fourth airbag gas delivery pipe 215D may be embedded in the pipe body 151 of the pipe 150. The end cap 160 is disposed at the end of the pipe 150 and has the end surface 160s, the tool channel 161 and a plurality of the airbag gas delivery pipe channels (for example, the first airbag gas delivery pipe channel 162A, the second airbag gas delivery pipe channel 162B, the third airbag gas delivery pipe channel 162C and the fourth airbag gas delivery pipe channel 162D), wherein the tool channel 161 and the airbag gas delivery pipe channels extend inwardly from the end surface 160s.

As illustrated in FIGS. 9A and 9B, the tool 105 enters and exits the end cap 160 through the tool channel 161. Each airbag gas delivery pipe is connected to the corresponding airbag through the corresponding airbag gas delivery pipe channel. For example, the first airbag gas delivery pipe 115A is connected to the first airbag 110A through the first airbag gas delivery pipe channel 162A, the second airbag gas delivery pipe 115B is connected to the second airbag 110B through the second airbag gas delivery pipe 162B, the third airbag gas delivery pipe 215C is connected to the third airbag 210C through the third airbag gas delivery pipe channel 162C, and the fourth airbag gas delivery pipe 215D is connected to the fourth airbag 210D through the fourth airbag gas delivery pipe channel 162D.

In this embodiment, the second gas supply module includes the gas second pressure source 231 and a plurality of control valves. The control valves of the second gas supply module of the present embodiment have the features same as or similar to the control valves of the second gas supply module 230 described above, and it will not be repeated again here. The number of control valves of the second gas supply module in the present embodiment is equal to the number of the airbags.

As illustrated in FIGS. 9A and 9C, the end cap 160 further has a plurality of the position-limiting grooves (for example, the first position-limiting groove 163A, the second position-limiting groove 163B, the third position-limiting groove 163C and a fourth position-limiting groove 163D). Each position-limiting groove communicates with the tool channel 161 and extends in a movement direction to provide the tool 105 with a degree of freedom of movement in the movement direction. For example, the first position-limiting groove 163A extends from the tool channel 161 toward +Y-axis to allow the tool 105 to bend along +Y-axis. The second position-limiting groove 163B extends from the tool channel 161 toward −X-axis to allow the tool 105 to bend along −X-axis. The third position-limiting groove 163C extends from the tool channel 161 toward +X-axis to allow the tool 105 to move along +X-axis. The fourth position-limiting groove 163D extends from the tool channel 161 toward −Y-axis to allow the tool 105 to move along −Y-axis.

In an embodiment, the radius (or volume) of the first airbag 210A, the radius (or volume) of the second airbag 210B, the radius (or volume) of the third airbag 210C, and/or the radius (or volume) of the fourth airbag 210D (or volume) to control the bending direction and/or the bending angle of the tool 105.

In summary, embodiments of the present disclosure provide a pipe support device which includes a pipe support module and a tool attitude control module. In an embodiment, the pipe support module includes a pipe, at least one expandable component and a first gas supply module, and the expandable component is disposed outside the pipe, for example, the the expandable component is connected to an outer wall of the pipe. After expansion, the expandable component may abut against (for example, directly press against) an inner wall of the cavity of the to-be-tested object. As a result, when the tool inserted into the pipe exerts a force on the inner wall of the cavity (for example, punctures), the expandable component abutting against (for example, directly pressing against) the inner wall of the cavity may resist a reaction force of the pipe to prevent the pipe support device from being deviating (for example, retreating or sliding). In addition, the first gas supply module may supply gas to the expandable component or release the gas within the expandable component to control the volume, size, shape and/or inner diameter of the expandable component. In addition, the controller may control the second gas supply module to supply gas with the corresponding gas pressure to the expandable component to achieve the aforementioned outer diameter control of the expandable component. In another embodiment, the tool attitude control module includes N airbags, a tool, a second gas supply module and a controller. N is a positive integer equal to or greater than 1. The tool is disposed among N airbags. The second gas supply module is connected to N airbags to supply N channels of gas to the N airbags respectively. For example, the second gas supply module includes N control valves which are respectively connected to N airbags to deliver gas to individual airbags and/or pump the gas out of individual airbag, thereby controlling or adjusting the radius (or volume) of individual airbag. As a result, by controlling the radius (or volume) of the N airbags, a bending direction and/or a bending angle of the tool may be adjusted (or controlled). In an embodiment, the controller may control the second gas supply module to supply gas with the corresponding pressure to the airbag to achieve the aforementioned airbag radius control. In an embodiment, a central angle between adjacent two of the N airbags is approximately 360/N (degrees). In other embodiment, the tool attitude control device further includes an end cap having N position-limiting slots, and the airbag may be located in the corresponding position-limiting groove. In an embodiment, the central angles between two adjacent airbags may be the same or different, and it may be achieved through the geometric shapes (e.g., extension direction) of the N position-limiting grooves of the end cap. Taking two airbags as an example, the second gas supply module may deliver the gas to individual airbags and/or pump the gas out of the individual airbag, thereby controlling or adjusting the radius (or volume) of the individual airbag for adjusting (or controlling) a bending angle of the tool in two axes (for example, two axes parallel to each other, but they not limited to this).

It will be apparent to those skilled in the art that various modifications and variations could be made to the disclosed embodiments. It is intended that the specification and examples be considered as exemplary only, with a true scope of the disclosure being indicated by the following claims and their equivalents.

What is claimed is:

1. A pipe support device, comprising:
a pipe;
an expandable component disposed outside the pipe;
a gas supply module connected to the expandable component and configured to deliver a gas to the expandable component or to pump the gas out of the expandable component; and
an expandable-component air pressure sensor connected to the expandable component and configured to sense a gas pressure of the gas of the expandable element.

2. The pipe support device according to claim 1, wherein the expandable component is an airbag.

3. The pipe support device according to claim 1, further comprising:
a plurality of airbags, wherein a tool is allowed to be disposed among the airbags;
an end cap having a tool channel and a plurality of airbag gas delivery pipe channels; and
a plurality of airbag air delivery pipes, wherein each airbag air delivery pipe is connected to the corresponding airbag;
wherein the tool enters and exits the end cap through the tool channel, and each airbag gas delivery pipe is connected to the corresponding airbag through the corresponding airbag gas delivery pipe channel.

4. The pipe support device according to claim 3, wherein the end cap is connected to the pipe, and the expandable component is spaced apart from the end cap by a distance.

5. The pipe support device according to claim 1, further comprising:
a controller electrically connected to the gas supply module and configured to:
control the gas supply module to supply the gas to the expandable component according to an inner diameter of a cavity.

6. The pipe support device according to claim 5, wherein the controller is further configured to:
determine whether the air pressure of the expandable component has reached a corresponding air pressure;
when the air pressure of the expandable element does not reach the corresponding air pressure, control the gas supply module to continue to supply the gas to the expandable element; and
when the air pressure of the expandable component reaches the corresponding air pressure, control the gas supply module to stop supplying the gas to the expandable component.

7. The pipe support device according to claim 1, wherein the pipe has a hollow channel, a tool has a piercing tip, the puncturing tip has an inclined surface, and the inclined surface and the expandable component are disposed on the same side.

8. The pipe support device according to claim 1, comprising:
two the expandable components oppositely disposed.

9. The pipe support device according to claim 1, wherein the expandable component has a connection surface, and the connection surface is connected to an outer wall surface of the pipe.

10. The pipe support device according to claim 1, wherein the pipe has a pipe body, and the pipe support device further comprising:
an expandable-component gas delivery pipe connected to the expandable component and partially embedded in the pipe body of the pipe.
